(19) United States
(12) Patent Application Publication
(10) Pub. No.: US 2025/0111509 A1
(43) Pub. Date: Apr. 3, 2025

(54) IMAGE PROCESSING APPARATUS, ENDOSCOPE, IMAGE PROCESSING METHOD, AND PROGRAM

(71) Applicant: FUJIFILM CORPORATION, Tokyo (JP)

(72) Inventor: Misaki GOTO, Kanagawa (JP)

(21) Appl. No.: 18/977,942

(22) Filed: Dec. 12, 2024

Related U.S. Application Data
(63) Continuation of application No. PCT/JP2023/025603, filed on Jul. 11, 2023.

(30) Foreign Application Priority Data
Aug. 24, 2022 (JP) 2022-133581

(51) Int. Cl. G06T 7/00; G16H 30/40

(52) U.S. Cl. CPC G06T 7/0012; G16H 30/40; G06T 2207/10068

(57) ABSTRACT

An image processing apparatus includes a processor. The processor is configured to acquire a plurality of medical images along a time series, the plurality of medical images depicting an observation target region; and perform image recognition processing on the plurality of medical images to detect a state change of the observation target region.

### CROSS-REFERENCE TO RELATED APPLICATIONS

This application is a continuation application of International Application No. PCT/JP2023/025603, filed Jul. 11, 2023, the disclosure of which is incorporated herein by reference in its entirety. Further, this application claims priority from Japanese Patent Application No. 2022-133581, filed Aug. 24, 2022, the disclosure of which is incorporated herein by reference in its entirety.

### BACKGROUND

### 1. Technical Field

The technology of the present disclosure relates to an image processing apparatus, an endoscope, an image processing method, and a program.

### 2. Related Art

JP2017-099509A discloses an endoscope processing apparatus. The endoscope processing apparatus receives, as a moving image, endoscopic images obtained by imaging the inside of a patient's body with an endoscope. A treatment implementation detection unit included in the endoscope processing apparatus detects a treatment tool used for endoscopic examination from frame images included in an endoscopic moving image by image recognition, and detects a treatment being implemented, based on the detected treatment tool. Further, the treatment implementation detection unit periodically searches for the treatment tool from within the frame images by image recognition.

JP2017-006337A discloses a medical support device including a detection unit that detects an event in a living body on the basis of a change in the temperature of an operating site or the surroundings of the operating site over time, and a notification information generation unit that generates notification information for providing a notification in accordance with the event in the living body detected by the detection unit. The detection unit detects the event in the living body on the basis of a change in a temperature distribution image of the operating site or the surroundings of the operating site over time. Further, the detection unit detects a state to be warned of as the event in the living body on the basis of a change in the temperature distribution image over time, and the notification information generation unit generates a warning image to provide a warning about the state.

### SUMMARY

An embodiment of the technology of the present disclosure provides an image processing apparatus, an endoscope, an image processing method, and a program that enable more accurate detection of a state change of an observation target region than detection of a state change of the observation target region using only a single medical image.

A first aspect according to the technology of the present disclosure is an image processing apparatus including a processor, the processor being configured to acquire a plurality of medical images along a time series, the plurality of medical images depicting an observation target region; and perform image recognition processing on the plurality of medical images to detect a state change of the observation target region.

A second aspect according to the technology of the present disclosure is the image processing apparatus according to the first aspect, in which the state change includes a change in adhesive color, a change in mucosal state including mucosal structure, and/or a change in mucus adhesion state.

A third aspect according to the technology of the present disclosure is the image processing apparatus according to the first aspect or the second aspect, in which in a case where the plurality of medical images are generated by an endoscope, the processor is configured to perform the image recognition processing based on an operation of the endoscope.

A fourth aspect according to the technology of the present disclosure is the image processing apparatus according to any one of the first to third aspects, in which the processor is configured to perform the image recognition processing based on a given medical instruction.

A fifth aspect according to the technology of the present disclosure is the image processing apparatus according to any one of the first to fourth aspects, in which the processor is configured to acquire region-of-interest information related to a region of interest included in the observation target region; and perform the image recognition processing based on the region-of-interest information.

A sixth aspect according to the technology of the present disclosure is the image processing apparatus according to the first to fifth aspects, in which the processor is configured to acquire site information related to a site corresponding to the observation target region; and perform the image recognition processing based on the site information.

A seventh aspect according to the technology of the present disclosure is the image processing apparatus according to any one of the first to sixth aspects, in which the processor is configured to start the image recognition processing in response to a first condition being satisfied.

An eighth aspect according to the technology of the present disclosure is the image processing apparatus according to the seventh aspect, in which in a case where the plurality of medical images are generated by an endoscope, the first condition includes a condition in which a tip portion of the endoscope has stopped moving or a condition in which a moving speed of the tip portion has decreased.

A ninth aspect according to the technology of the present disclosure is the image processing apparatus according to the seventh aspect or the eighth aspect, in which the first condition includes a condition in which an instruction to start the image recognition processing is given.

A tenth aspect according to the technology of the present disclosure is the image processing apparatus according to any one of the seventh to ninth aspects, in which the first condition includes a condition in which a region of interest is included in the observation target region.

An eleventh aspect according to the technology of the present disclosure is the image processing apparatus according to any one of the seventh to tenth aspects, in which the first condition includes a condition in which a site corresponding to the observation target region is a site designated as an observation target.

A twelfth aspect according to the technology of the present disclosure is the image processing apparatus according to any one of the first to eleventh aspects, in which the processor is configured to end the image recognition processing in response to a second condition being satisfied.

A thirteenth aspect according to the technology of the present disclosure is the image processing apparatus according to the twelfth aspect, in which the processor is configured to delete first information that is information based on the image recognition processing, in response to the second condition being satisfied.

A fourteenth aspect according to the technology of the present disclosure is the image processing apparatus according to the thirteenth aspect, in which the first information is held during a period from a start of the image recognition processing to an end of the image recognition processing, and the processor is configured to delete the first information in response to the end of the image recognition processing.

A fifteenth aspect according to the technology of the present disclosure is the image processing apparatus according to any one of the twelfth to fourteenth aspects, in which in a case where the plurality of medical images are generated by an endoscope, the second condition includes a condition in which a tip portion of the endoscope has started moving or a condition in which a moving speed of the tip portion has increased.

A sixteenth aspect according to the technology of the present disclosure is the image processing apparatus according to any one of the twelfth to fifteenth aspects, in which the second condition includes a condition in which an instruction to end the image recognition processing is given.

A seventeenth aspect according to the technology of the present disclosure is the image processing apparatus according to any one of the twelfth to sixteenth aspects, in which the second condition includes a condition in which a region of interest is not included in the observation target region.

An eighteenth aspect according to the technology of the present disclosure is the image processing apparatus according to any one of the twelfth to seventeenth aspects, in which the second condition includes a condition in which a site corresponding to the observation target region is a site different from a site designated as an observation target.

A nineteenth aspect according to the technology of the present disclosure is the image processing apparatus according to any one of the first to eighteenth aspects, in which in a case where the plurality of medical images are generated by an endoscope, the processor is configured to detect the state change based on an operation of the endoscope.

A twentieth aspect according to the technology of the present disclosure is the image processing apparatus according to any one of the first to nineteenth aspects, in which in a case where the plurality of medical images are generated by an endoscope and a fluid is delivered from the endoscope into a body including the observation target region, the processor is configured to acquire fluid delivery information related to a delivery of the fluid; and detect the state change based on the fluid delivery information.

A twenty-first aspect according to the technology of the present disclosure is the image processing apparatus according to any one of the first to twentieth aspects, in which the processor is configured to detect the state change based on a given medical instruction.

A twenty-second aspect according to the technology of the present disclosure is the image processing apparatus according to any one of the first to twenty-first aspects, in which the processor is configured to acquire region-of-interest information related to a region of interest included in the observation target region; and detect the state change on condition that the region-of-interest information is acquired.

A twenty-third aspect according to the technology of the present disclosure is the image processing apparatus according to any one of the first to twenty-second aspects, in which the processor is configured to acquire site information related to a site corresponding to the observation target region; and detect the state change on condition that the site information is acquired.

A twenty-fourth aspect according to the technology of the present disclosure is the image processing apparatus according to any one of the first to twenty-third aspects, in which the processor is configured to derive lesion information related to a lesion in the observation target region, based on the state change.

A twenty-fifth aspect according to the technology of the present disclosure is the image processing apparatus according to the twenty-fourth aspect, in which the observation target region includes a region of interest, the state change includes a change in the region of interest, and the change in the region of interest is a change from a state in which mucus adheres to the region of interest to a state in which a non-neoplastic polyp appears in the region of interest.

A twenty-sixth aspect according to the technology of the present disclosure is an image processing apparatus according to a twenty-fourth aspect, in which the observation target region includes a region of interest, and in a case where the plurality of medical images are generated by an endoscope and a fluid is delivered from the endoscope into a body including the observation target region, the state change includes a change in the region of interest caused by a delivery of the fluid, and the processor is configured to acquire delivery amount information indicating a delivery amount of the fluid; and derive the lesion information based on the state change and the delivery amount information.

A twenty-seventh aspect according to the technology of the present disclosure is the image processing apparatus according to the twenty-sixth aspect, in which the change in the region of interest is a change from a state in which mucus adheres to the region of interest to a state in which a non-neoplastic polyp appears in the region of interest.

A twenty-eighth aspect according to the technology of the present disclosure is the image processing apparatus according to any one of the twenty-fourth to twenty-seventh aspects, in which in a case where the plurality of medical images are generated by an endoscope, the processor is configured to derive the lesion information at a timing determined based on an operation of the endoscope.

A twenty-ninth aspect according to the technology of the present disclosure is the image processing apparatus according to any one of the twenty-fourth to twenty-eighth aspects, in which the processor is configured to derive the lesion information at a timing determined based on a given medical instruction.

A thirtieth aspect according to the technology of the present disclosure is the image processing apparatus according to any one of the twenty-fourth to twenty-ninth aspects, in which in a case where the lesion information is derived, the processor is configured to determine the lesion information in accordance with a given determination instruction.

A thirty-first aspect according to the technology of the present disclosure is the image processing apparatus according to any one of the twenty-fourth to thirtieth aspects, in which the processor is configured to acquire region-of-interest information related to a region of interest included in the observation target region; and derive the lesion information in a case where the region-of-interest information is information related to a specific region of interest.

A thirty-second aspect according to the technology of the present disclosure is the image processing apparatus according to any one of the twenty-fourth to thirty-first aspects, in which the processor is configured to acquire site information related to a site corresponding to the observation target region; and derive the lesion information in a case where the site information is information related to a specific site.

A thirty-third aspect according to the technology of the present disclosure is the image processing apparatus according to any one of the first to thirty-second aspects, in which the processor is configured to output second information that is information based on the image recognition processing.

A thirty-fourth aspect according to the technology of the present disclosure is the image processing apparatus according to the thirty-third aspect, in which an output destination of the second information is a display device, and the display device is configured to display the second information.

A thirty-fifth aspect according to the technology of the present disclosure is an endoscope including the image processing apparatus according to any one of the first to thirty-fourth aspects, and an endoscope main body to be inserted into a body including the observation target region.

A thirty-sixth aspect according to the technology of the present disclosure is an image processing method including acquiring a plurality of medical images along a time series, the plurality of medical images depicting an observation target region; and performing image recognition processing on the plurality of medical images to detect a state change of the observation target region.

A thirty-seventh aspect according to the technology of the present disclosure is a program for causing a computer to execute processing including acquiring a plurality of medical images along a time series, the plurality of medical images depicting an observation target region; and performing image recognition processing on the plurality of medical images to detect a state change of the observation target region.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of the endoscope system;

FIG. 3 is a block diagram illustrating an example hardware configuration of an electric system of the endoscope system;

FIG. 4 is a block diagram illustrating an example of functions of main components of a processor of a control device included in an endoscope;

FIG. 5 is a conceptual diagram illustrating an example of a correlation among a camera, an NVM, a reception device, an image acquisition unit, a site detection unit, a region-of-interest detection unit, and a control unit;

FIG. 6 is a conceptual diagram illustrating an example of a correlation among the camera, the image acquisition unit, and an endoscope detection unit;

FIG. 7 is a conceptual diagram illustrating an example of a correlation among the camera, the reception device, the image acquisition unit, the site detection unit, the region-of-interest detection unit, the endoscope detection unit, a state change detection unit, and the control unit;

FIG. 8 is a conceptual diagram illustrating an example of a correlation among the state change detection unit, a lesion information derivation unit, the control unit, and a display device;

FIG. 9A is a flowchart illustrating an example of the flow of a medical support process;

FIG. 9B is a continuation of the flowchart illustrated in FIG. 9A;

FIG. 10 is a block diagram illustrating a modification of output timings of a start instruction signal and an end instruction signal;

FIG. 11 is a block diagram illustrating an example of a modification of the lesion information derivation unit;

FIG. 12 is a flowchart illustrating a first modification of the flow of the medical support process; and

FIG. 13 is a flowchart illustrating a second modification of the flow of the medical support process.

### DETAILED DESCRIPTION

An example of an image processing apparatus, an endoscope, an image processing method, and a program according to an embodiment of the technology of the present disclosure will be described hereinafter with reference to the accompanying drawings.

First, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “ELectro-luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”. FIFO is an abbreviation for “First In First Out”.

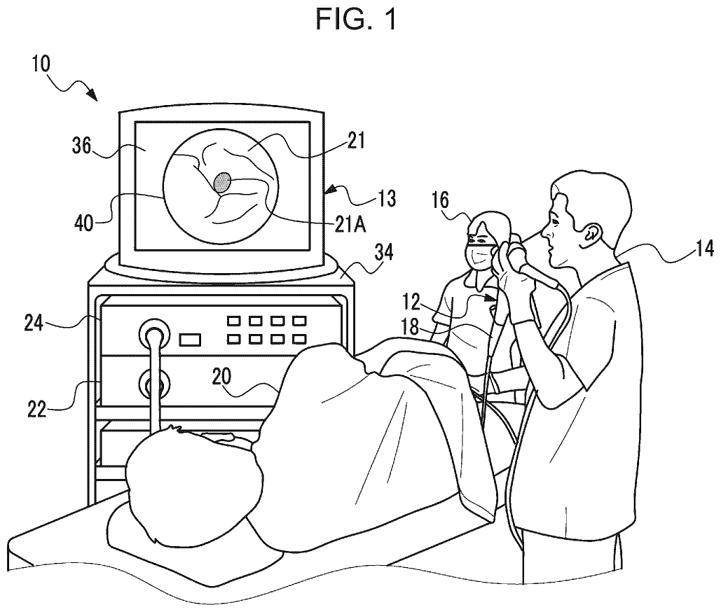

As an example, as illustrated in FIG. 1, an endoscope system 10 includes an endoscope 12 and a display device 13. The endoscope 12 is used by a doctor 14 for endoscopic examination. At least one support staff member 16 (for example, a nurse or the like) supports the doctor 14 in the endoscopic examination. In the following description, the doctor 14 and the support staff member 16 are also referred to as “users” without reference numerals unless otherwise distinguished.

The endoscope 12 includes an endoscope main body 18. The endoscope 12 is a device for performing medical care in an observation target region 21 included in the body (here, the inside of the large intestine, as an example) of a subject 20 (for example, a patient) by using the endoscope main body 18. The observation target region 21 is a region to be observed by the doctor 14. The endoscope main body 18 is inserted into the body of the subject 20. In the endoscope 12, the endoscope main body 18 inserted into the body of the subject 20 performs imaging of the observation target region 21 in the body of the subject 20, and performs various medical treatments on the observation target region 21 as necessary. The endoscope 12 is an example of an “endoscope” according to the technology of the present disclosure. The endoscope main body 18 is an example of an “endoscope main body” according to the technology of the present disclosure.

The endoscope 12 performs imaging of the inside of the body of the subject 20 to acquire an image indicating the condition of the inside of the body, and outputs the acquired image. In the example illustrated in FIG. 1, an endoscope for lower endoscopy is illustrated as an example of the endoscope 12. The endoscope for lower endoscopy is merely an example, and the technology of the present disclosure also remains applicable when the endoscope 12 is any other type of endoscope such as an upper digestive tract endoscope or a bronchial endoscope.

In this embodiment, the endoscope 12 is an endoscope having an optical imaging function of capturing an image of reflected light obtained by irradiating the inside of the body with light and reflecting the light from the observation target region 21. However, this is merely an example, and the technology of the present disclosure also remains applicable even when the endoscope 12 is an ultrasonic endoscope. The technology of the present disclosure also remains applicable even when a modality that generates a plurality of frames for examination or surgery (for example, a moving image obtained by imaging using X-rays or the like, or a moving image based on a reflected wave of an ultrasound wave radiated from outside the body of the subject 20) is used instead of the endoscope 12.

The endoscope 12 includes a control device 22 and a light source device 24. The control device 22 and the light source device 24 are installed in a cart 34. The cart 34 is provided with a plurality of shelves arranged in the vertical direction, and the control device 22 and the light source device 24 are installed on a lower shelf and an upper shelf. The display device 13 is installed on top of the cart 34.

The display device 13 displays various kinds of information including images. Examples of the display device 13 include a liquid crystal display and an EL display. The display device 13 shows one or more screens side by side. In the example illustrated in FIG. 1, a screen 36 is illustrated. A tablet terminal with a display may be used instead of or together with the display device 13.

The screen 36 displays an endoscopic image 40 obtained by the endoscope 12. The endoscopic image 40 depicts the observation target region 21 including a region of interest 21A. The region of interest 21A is a region determined as a region requiring observation in the observation target region 21 (for example, a region determined as a region of interest to the doctor 14 to differentiate a lesion). The endoscopic image 40 is an image generated by the endoscope 12 imaging the observation target region 21 in the body of the subject 20. Examples of the observation target region 21 include the inner wall of the large intestine. The inner wall of the large intestine is merely an example, and the observation target region 21 is any region that can be imaged by the endoscope 12. Examples of the region that can be imaged by the endoscope 12 include the inner wall or the outer wall of a luminal organ. Examples of the luminal organ include the small intestine, the duodenum, the esophagus, the stomach, and the bronchus. The observation target region 21 to be imaged by the endoscope 12 is an example of an “observation target region” according to the technology of the present disclosure. The endoscopic image 40 is an example of a “medical image” according to the technology of the present disclosure.

The endoscopic image 40 displayed on the screen 36 is one frame included in a moving image that includes a plurality of frames. That is, the screen 36 displays endoscopic images 40 of the plurality of frames at a specified frame rate (for example, several tens of frames/second).

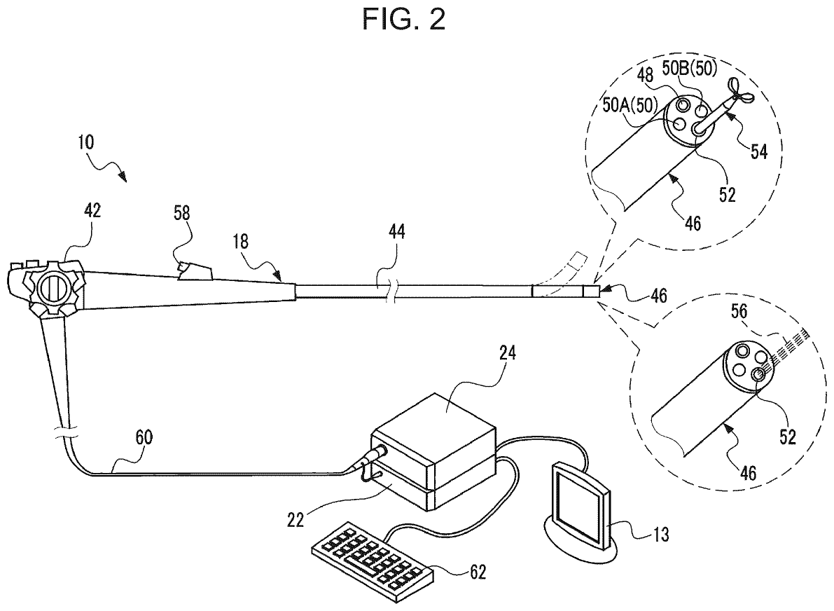

As an example, as illustrated in FIG. 2, the endoscope 12 includes an operation section 42 and an insertion section 44. The insertion section 44 is partially bent in response to the operation section 42 being operated. When the doctor 14 operates the operation section 42, the insertion section 44 is inserted while being bent in accordance with the shape of the inside of the body of the subject 20 (for example, the shape of the large intestine).

The insertion section 44 has a tip portion 46 provided with a camera 48, an illumination device 50, and a treatment opening 52. The camera 48 is a device that performs imaging of the inside of the body of the subject 20. Examples of the camera 48 include a CMOS camera. However, this is merely an example, and the camera 48 may be any other type of camera such as a CCD camera. The illumination device 50 has illumination windows 50A and 50B. The illumination device 50 emits light through the illumination windows 50A and 50B. Examples of the type of light emitted from the illumination device 50 include visible light (for example, white light or the like) and non-visible light (for example, near-infrared light or the like). The illumination device 50 further emits special light through the illumination windows 50A and 50B. Examples of the special light include light for BLI and/or light for LCI. The camera 48 performs imaging of the inside of the body of the subject 20 using an optical method, with the inside of the body of the subject 20 irradiated with light from the illumination device 50.

The treatment opening 52 is used as a treatment tool protruding port through which a treatment tool 54 protrudes from the tip portion 46, a suction port through which blood, bodily waste, and so on are sucked, and a delivery port through which a fluid 56 is delivered.

The treatment tool 54 protrudes from the treatment opening 52 in accordance with the operation of the doctor 14. The treatment tool 54 is inserted into the insertion section 44 from a treatment tool insertion port 58. The treatment tool 54 passes through the insertion section 44 and protrudes into the body of the subject 20 from the treatment opening 52. In the example illustrated in FIG. 2, forceps protrude from the treatment opening 52 as the treatment tool 54. The forceps are merely an example of the treatment tool 54, and other examples of the treatment tool 54 include a wire, a scalpel, and an ultrasound probe. The tip portion 46 and the treatment tool 54 are examples of a “tip portion of an endoscope” according to the technology of the present disclosure.

A suction pump (not illustrated) is connected to the endoscope main body 18, and blood, bodily waste, and so on are sucked through the treatment opening 52 by the suction force of the suction pump. The suction force of the suction pump is controlled in accordance with an instruction given from the doctor 14 to the endoscope 12 via the operation section 42 or the like. A supply pump (not illustrated) is connected to the endoscope main body 18, and the fluid 56 (for example, a gas and a liquid) is supplied into the endoscope main body 18 by the supply pump. The fluid 56 supplied from the supply pump to the endoscope main body 18 is delivered through the treatment opening 52. In accordance with an instruction given from the doctor 14 to the endoscope 12 via the operation section 42 or the like, a gas (for example, air) and a liquid (for example, a normal saline solution) are selectively delivered as the fluid 56 into the body from the treatment opening 52. The delivery amount of the fluid 56 is controlled in accordance with an instruction given from the doctor 14 to the endoscope 12 via the operation section 42 or the like.

The endoscope main body 18 is connected to the control device 22 and the light source device 24 via a universal cord 60. The control device 22 is connected to the display device 13 and a reception device 62. The reception device 62 receives an instruction from the user and outputs the received instruction as an electrical signal. In the example illustrated in FIG. 2, an example of the reception device 62 is a keyboard. However, this is merely an example, and the reception device 62 may be a mouse, a touch panel, a foot switch, a microphone, and/or the like.

The control device 22 controls the entire endoscope 12. For example, the control device 22 controls the light source device 24, transmits and receives various signals to and from the camera 48, and displays various kinds of information on the display device 13. The light source device 24 emits light under the control of the control device 22 and supplies the light to the illumination device 50. The illumination device 50 incorporates a light guide, and the light supplied from the light source device 24 is emitted from the illumination windows 50A and 50B via the light guide. The control device 22 controls the camera 48 to perform imaging, acquires the endoscopic image 40 (see FIG. 1) from the camera 48, and outputs the endoscopic image 40 to a specified output destination (for example, the display device 13).

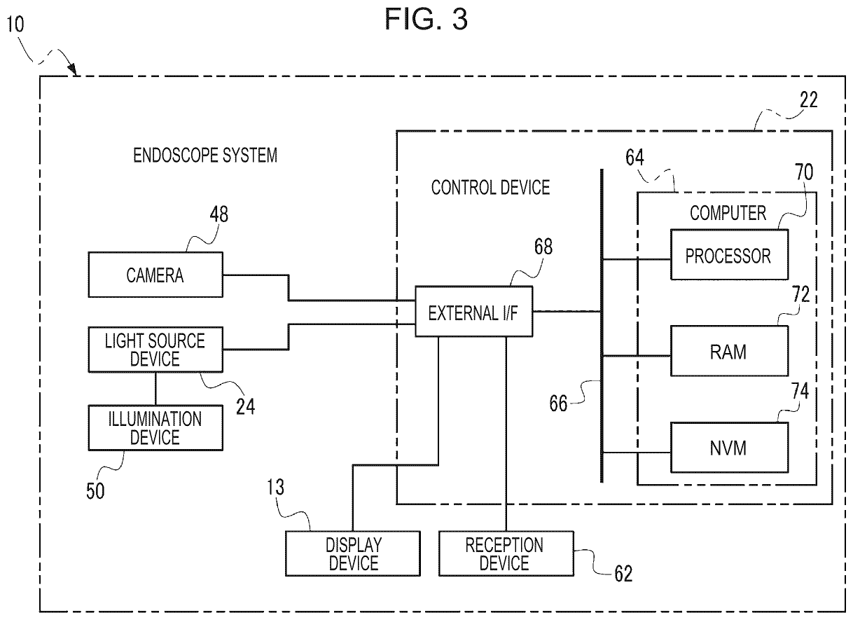

As an example, as illustrated in FIG. 3, the control device 22 includes a computer 64. The computer 64 is an example of an “image processing apparatus” and a “computer” according to the technology of the present disclosure. The computer 64 includes a processor 70, a RAM 72, and an NVM 74. The processor 70, the RAM 72, and the NVM 74 are electrically connected to each other. The processor 70 is an example of a “processor” according to the technology of the present disclosure.

The control device 22 includes the computer 64, a bus 66, and an external I/F 68. The computer 64 includes the processor 70, the RAM 72, and the NVM 74. The processor 70, the RAM 72, the NVM 74, and the external I/F 68 are connected to the bus 66.

For example, the processor 70 has a CPU and a GPU, and controls the entire control device 22. The GPU operates under the control of the CPU and is responsible for performing various types of graphics-based processing, arithmetic operations using a neural network, and the like. The processor 70 may include one or more CPUs with integrated GPU functions, or may include one or more CPUs without integrated GPU functions.

The RAM 72 is a memory that temporarily stores information, and is used as a work memory by the processor 70. The NVM 74 is a non-volatile storage device that stores various programs, various parameters, and the like. Examples of the NVM 74 include a flash memory (for example, an EEPROM and/or an SSD). The flash memory is merely an example, and the NVM 74 may be any other non-volatile storage device such as an HDD, or a combination of two or more types of non-volatile storage devices.

The external I/F 68 manages transmission and reception of various kinds of information between a device external to the control device 22 (hereinafter also referred to as an “external device”) and the processor 70. Examples of the external I/F 68 include a USB interface.

The camera 48 is connected to the external I/F 68, as one of such external devices, and the external I/F 68 manages transmission and reception of various kinds of information between the camera 48 and the processor 70. The processor 70 controls the camera 48 via the external I/F 68. Further, the processor 70 acquires the endoscopic image 40 (see FIG. 1), which is obtained by imaging the inside of the body of the subject 20 using the camera 48, via the external I/F 68.

The light source device 24 is connected to the external I/F 68, as one of the external devices, and the external I/F 68 manages transmission and reception of various kinds of information between the light source device 24 and the processor 70. The light source device 24 supplies light to the illumination device 50 under the control of the processor 70. The illumination device 50 emits the light supplied from the light source device 24.

The display device 13 is connected to the external I/F 68, as one of the external devices, and the processor 70 controls the display device 13 via the external I/F 68 to display various kinds of information on the display device 13.

The reception device 62 is connected to the external I/F 68, as one of the external devices, and the processor 70 acquires an instruction received by the reception device 62 via the external I/F 68 and performs processing corresponding to the acquired instruction.

In recent years, a technique for detecting the presence or absence of a lesion or identifying the type of the lesion through AI image recognition processing performed by a modality such as the endoscope 12 has become increasingly widespread. This technique enables detection of the presence or absence of a lesion or identification of the type of the lesion from an image recognition result obtained by performing AI image recognition processing only on a single frame. Such a method causes blurring in image recognition results between frames, and involves post-processing to reduce the blurring, such as averaging image recognition results obtained in a time-series manner. In actuality, however, the doctor 14 differentiates a lesion by comprehensively determining the site being observed, the change in mucus state caused by the fluid 56, the change in mucosal state caused by the fluid 56, and/or the like.

For example, it is known that a sessile serrated lesion, which is a type of colonic neoplastic polyp, is a polyp that is difficult to visually distinguish from a hyperplastic polyp and is likely to become cancerous, and it is necessary to carefully differentiate between a sessile serrated lesion and a hyperplastic polyp by comprehensively determining various kinds of information obtained in a time series from the endoscope 12. For example, a sessile serrated lesion has a feature of mucus adhering to a polyp and a feature of being likely to occur in the right large intestine (for example, the ascending colon, the transverse colon, and the like). Accordingly, it is important for the doctor 14 to capture these features as materials for determination without overlooking them to differentiate the lesion.

Barrett's esophagus-associated cancer is also known as one of the lesions that are difficult to differentiate, like sessile serrated lesions. Barrett's esophagus-associated cancer also occurs frequently at the junction of the stomach and the esophagus and thus is difficult to observe or causes a few changes (i.e., bulging, change in color, and the like) from the surrounding mucosa. The doctor 14 differentiates Barrett's esophagus-associated cancer by carefully observing Barrett's mucosa and then comprehensively considering time-series state changes of the mucosa caused by changing the state of air supply from the endoscope 12 (for example, state changes in which the mucosa is flat with air supply and bulges without air supply).

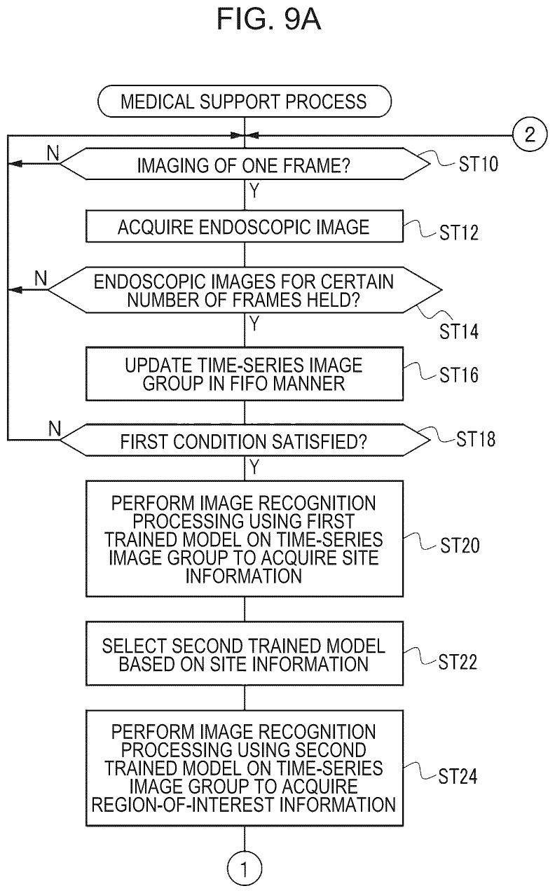

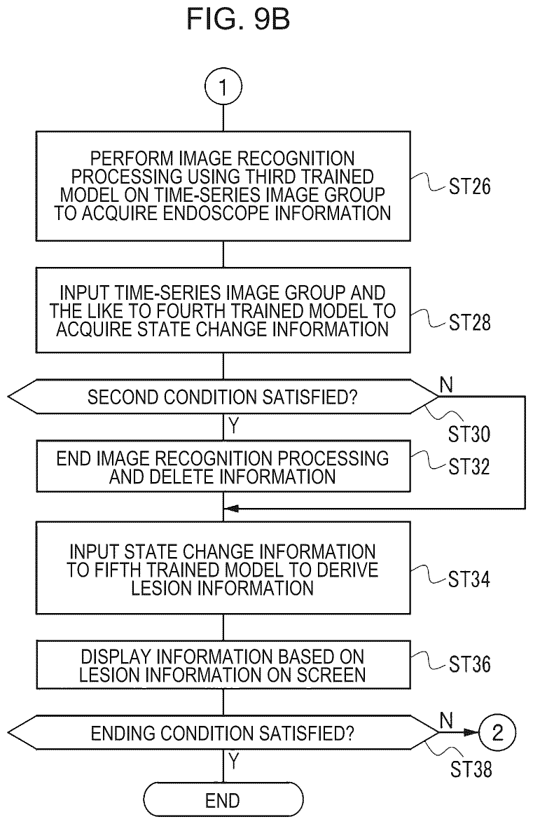

In view of such circumstances, in this embodiment, the processor 70 of the control device 22 performs a medical support process to support the doctor 14 in differentiation of lesions (see FIGS. 9A and 9B). The medical support process includes a process of acquiring a plurality of endoscopic images 40 along a time series, the plurality of endoscopic images 40 depicting the observation target region 21, and performing image recognition processing on the plurality of endoscopic images 40 to detect a state change of the observation target region 21.

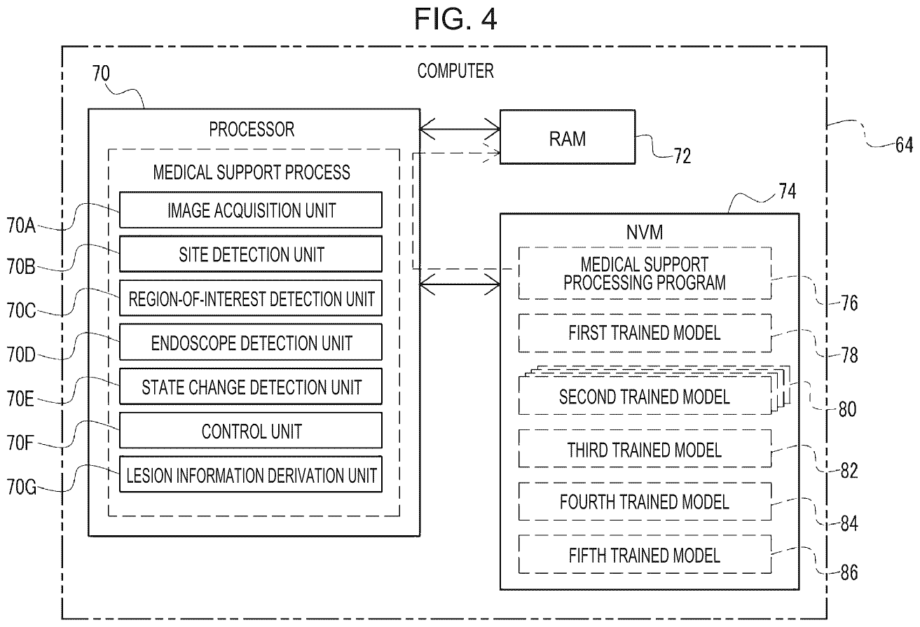

As an example, as illustrated in FIG. 4, the NVM 74 stores a medical support processing program 76. The medical support processing program 76 is an example of a “program” according to the technology of the present disclosure. The processor 70 reads the medical support processing program 76 from the NVM 74 and executes the read medical support processing program 76 on the RAM 72. The medical support process is implemented by the processor 70 operating as an image acquisition unit 70A, a site detection unit 70B, a region-of-interest detection unit 70C, an endoscope detection unit 70D, a state change detection unit 70E, a control unit 70F, and a lesion information derivation unit 70G in accordance with the medical support processing program 76 executed on the RAM 72.

The NVM 74 stores a first trained model 78, a plurality of second trained models 80, a third trained model 82, a fourth trained model 84, and a fifth trained model 86. The first trained model 78, the plurality of second trained models 80, the third trained model 82, the fourth trained model 84, and the fifth trained model 86 are all optimized through machine learning performed for a neural network in advance.

The first trained model 78 is used by the site detection unit 70B. The plurality of second trained models 80 are selectively used by the region-of-interest detection unit 70C. The third trained model 82 is used by the endoscope detection unit 70D. The fourth trained model 84 is used by the state change detection unit 70E. The fifth trained model 86 is used by the lesion information derivation unit 70G.

In the following description, the first trained model 78, the plurality of second trained models 80, the third trained model 82, the fourth trained model 84, and the fifth trained model 86 are referred to as “trained models”, for convenience of description, unless otherwise distinguished. In the following description, furthermore, processing using a trained model is described as processing that is actively performed mainly by the trained model, for convenience of description. That is, for convenience of description, the trained model is described as a function of performing processing on input information and outputting a processing result.

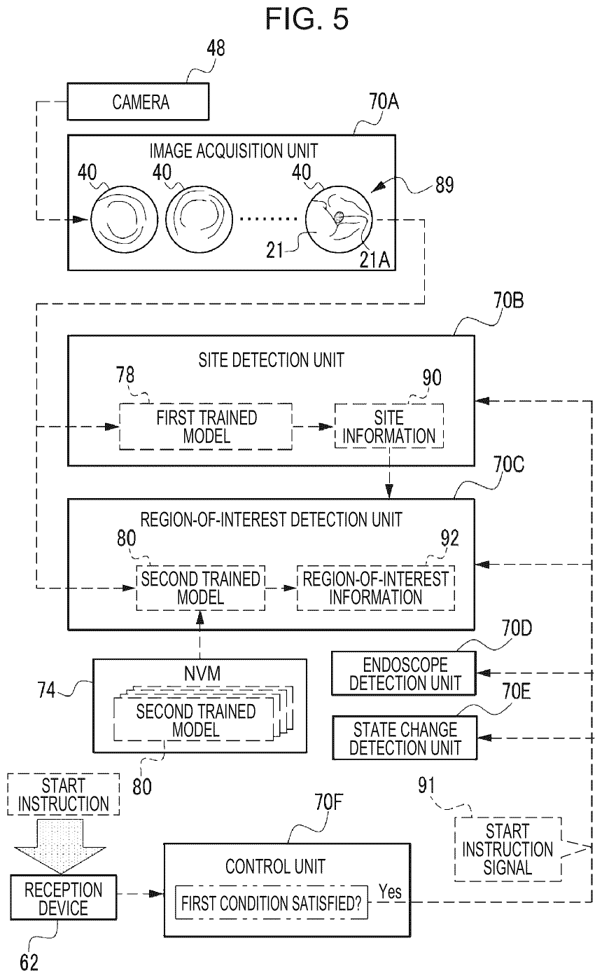

As an example, as illustrated in FIG. 5, the image acquisition unit 70A acquires, from the camera 48, the endoscopic images 40 generated through imaging performed by the camera 48 in accordance with an imaging frame rate (for example, several tens of frames/second), on a frame-by-frame basis.

The image acquisition unit 70A holds a time-series image group 89. The time-series image group 89 is a plurality of time-series endoscopic images 40 in which the observation target region 21 appears. The time-series image group 89 includes, for example, endoscopic images 40 for a certain number of frames (for example, a predetermined number of frames in the range of several tens to several hundreds of frames). The image acquisition unit 70A updates the time-series image group 89 in a FIFO manner each time an endoscopic image 40 is acquired from the camera 48.

While an example configuration is presented in which the image acquisition unit 70A holds and updates the time-series image group 89, this is merely an example. For example, the time-series image group 89 may be held and updated in a memory connected to the processor 70, such as the RAM 72.

When a first condition is satisfied, the control unit 70F outputs a start instruction signal 91 to the site detection unit 70B, the region-of-interest detection unit 70C, the endoscope detection unit 70D, and the state change detection unit 70E. When the start instruction signal 91 is input from the control unit 70F, the site detection unit 70B, the region-of-interest detection unit 70C, the endoscope detection unit 70D, and the state change detection unit 70E start AI image recognition processing. The AI image recognition processing refers to, for example, image recognition processing performed by the site detection unit 70B using the first trained model 78, image recognition processing performed by the region-of-interest detection unit 70C using the second trained model 80, image recognition processing performed by the endoscope detection unit 70D using the third trained model 82, and image recognition processing performed by the state change detection unit 70E using the fourth trained model 84.

Examples of the first condition include conditions including a condition in which a start instruction is given to the endoscope 12 (in the example illustrated in FIG. 5, a condition in which the start instruction is received by the reception device 62). The start instruction refers to an instruction to start the AI image recognition processing.

The site detection unit 70B performs the image recognition processing using the first trained model 78 on the time-series image group 89 (i.e., the plurality of time-series endoscopic images 40 held by the image acquisition unit 70A) to detect a site in the subject 20. The first trained model 78 is a trained model for AI-based object detection, and is optimized through machine learning performed for a neural network using first labeled data. Examples of the first labeled data include labeled data in which a plurality of images obtained in a time series by imaging a site that may be a target for endoscopic examination (for example, a plurality of images corresponding to the plurality of time-series endoscopic images 40) are set as example data and site information 90 related to the site is set as ground-truth data. While an example configuration is presented in which only one first trained model 78 is used by the site detection unit 70B, this is merely an example. For example, the site detection unit 70B may use a first trained model 78 selected from among a plurality of first trained models 78. In this case, desirably, the first trained models 78 are created through machine learning specific to the respective types of endoscopic examination, and the first trained model 78 corresponding to the type of the endoscopic examination currently being performed is selected and used by the site detection unit 70B.

Examples of the site information 90 include information indicating the name of the site. Examples of the site that may be the target for endoscopic examination include a single site included in the large intestine (for example, the ascending colon) and a plurality of adjacent sites (for example, the sigmoid colon and the descending colon). While sites in the large intestine are used here as sites that may be the target for endoscopic examination, this is merely an example, and the site may be a site in a luminal organ such as the stomach, the esophagus, the gastroesophageal junction, the duodenum, or the bronchus.

The site detection unit 70B acquires the time-series image group 89 and inputs the acquired time-series image group 89 to the first trained model 78. Accordingly, the first trained model 78 outputs site information 90 corresponding to the plurality of input endoscopic images 40. The site detection unit 70B acquires the site information 90 output from the first trained model 78. The site information 90 acquired by the site detection unit 70B is information related to a site corresponding to the observation target region 21 depicted in the endoscopic images 40. The site information 90 is an example of “site information” and “first information that is information based on the image recognition processing” according to the technology of the present disclosure.

The region-of-interest detection unit 70C performs the image recognition processing using the second trained model 80 on the time-series image group 89 to detect the region of interest 21A included in the observation target region 21. Examples of the region of interest 21A include a mucus region (i.e., a region to which mucus adheres), Barrett's mucosa, and/or Barrett's adenocarcinoma.

The second trained model 80 used by the region-of-interest detection unit 70C is selected from among the plurality of second trained models 80 stored in the NVM 74. The plurality of second trained models 80 stored in the NVM 74 correspond to different sites. The region-of-interest detection unit 70C selects, from among the plurality of second trained models 80, a second trained model 80 corresponding to the site information 90 acquired by the site detection unit 70B and uses the selected second trained model 80. For example, when the site information 90 is information related to the ascending colon, a second trained model 80 created for the ascending colon is selected from among the plurality of second trained models 80 by the region-of-interest detection unit 70C. For example, when the site information 90 is information related to the sigmoid colon and the descending colon, a second trained model 80 created for the sigmoid colon and the descending colon is selected from among the plurality of second trained models 80 by the region-of-interest detection unit 70C. While an example configuration is presented in which the second trained model 80 to be used by the region-of-interest detection unit 70C is selected from among the plurality of second trained models 80 in accordance with the site information 90, this is merely an example. For example, a single second trained model 80 corresponding to all the sites that may be targets for endoscopic examination may be determined in advance as the second trained model 80 to be used by the region-of-interest detection unit 70C.

The second trained model 80 is a trained model for AI-based object detection, and is optimized through machine learning performed for a neural network using second labeled data. Examples of the second labeled data include labeled data in which a plurality of images (for example, a plurality of images corresponding to a plurality of time-series endoscopic images 40) obtained in a time series by imaging a region of interest 21A that may be a target for endoscopic examination (for example, a region of interest 21A occurring in a site identified from the site information 90) are set as example data and region-of-interest information 92 related to the region of interest 21A is set as ground-truth data. Examples of the region-of-interest information 92 include information indicating the presence or absence of the region of interest 21A and information indicating the name of the region of interest 21A.

The region-of-interest detection unit 70C acquires the time-series image group 89 and inputs the acquired time-series image group 89 to the second trained model 80. Accordingly, the second trained model 80 outputs region-of-interest information 92 corresponding to the input time-series image group 89. The region-of-interest detection unit 70C acquires the region-of-interest information 92 output from the second trained model 80. The region-of-interest information 92 acquired by the region-of-interest detection unit 70C is information related to the region of interest 21A included in the observation target region 21 depicted in the endoscopic images 40. The region-of-interest information 92 is an example of “region-of-interest information” and “first information that is information based on the image recognition processing” according to the disclosed technique.

Examples of the region-of-interest information 92 include information indicating the name of the region of interest 21A. Examples of the region of interest 21A that may be the target for endoscopic examination include a single region of interest 21A (for example, a specific mucus region) and a plurality of regions of interest 21A that transition in a time series (for example, a specific mucus region and a specific mucosal region).

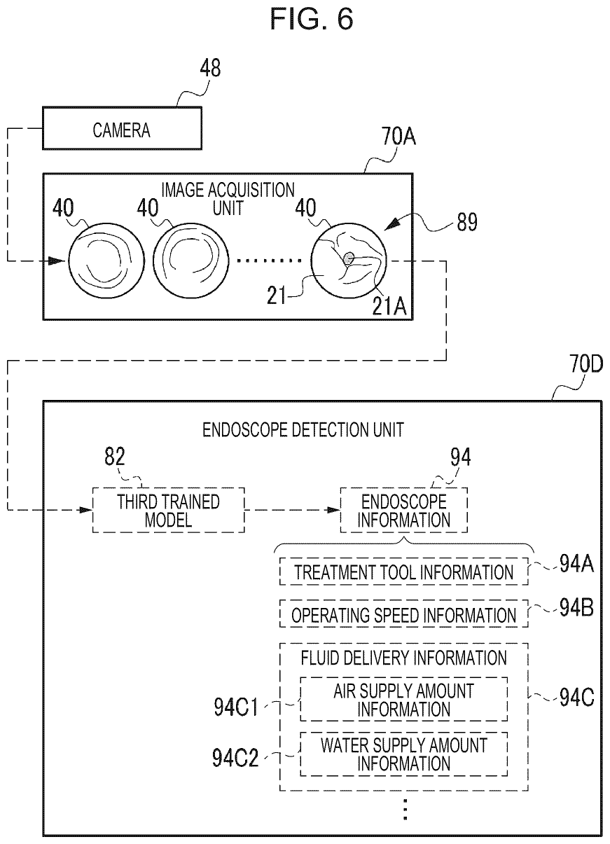

As an example, as illustrated in FIG. 6, the endoscope detection unit 70D performs the image recognition processing using the third trained model 82 on the time-series image group 89 to detect the operation of the endoscope 12. The third trained model 82 is a trained model for AI-based object detection, and is optimized through machine learning performed for a neural network using third labeled data. Examples of the third labeled data include labeled data in which a plurality of images obtained in a time series by imaging the inside of the body using the camera 48 are set as example data and endoscope information 94 related to the operation of the endoscope 12 is set as ground-truth data. While an example configuration is presented in which the endoscope detection unit 70D uses only a single third trained model 82, this is merely an example. For example, the endoscope detection unit 70D may use a third trained model 82 selected from among a plurality of third trained models 82. In this case, desirably, the third trained models 82 are created through machine learning specific to the respective types of endoscopic examination, and the third trained model 82 corresponding to the type of the endoscopic examination currently being performed (here, the type of the endoscope 12, as an example) is selected and used by the endoscope detection unit 70D.

The endoscope detection unit 70D acquires the time-series image group 89 and inputs the acquired time-series image group 89 to the third trained model 82. Accordingly, the third trained model 82 outputs endoscope information 94 corresponding to the plurality of input endoscopic images 40. The endoscope detection unit 70D acquires the endoscope information 94 output from the third trained model 82. The endoscope information 94 acquired by the endoscope detection unit 70D is information related to the operation of the endoscope 12 currently in use. The endoscope information 94 is an example of “first information that is information based on the image recognition processing” according to the technology of the present disclosure.

Examples of the endoscope information 94 include treatment tool information 94A, operating speed information 94B, and fluid delivery information 94C. The treatment tool information 94A is information related to the treatment tool 54 (see FIG. 2). Examples of the information related to the treatment tool 54 include information indicating whether the treatment tool 54 is in use, and information indicating the type of the treatment tool 54 in use. The operating speed information 94B is information related to the operating speed of the tip portion 46 (see FIG. 2) of the endoscope 12 (for example, information related to a speed expressed in the unit “millimeter/second”).

The fluid delivery information 94C is information related to delivery of the fluid 56 (see FIG. 2). The information related to delivery of the fluid 56 refers to, for example, information related to the amount of the fluid 56 delivered per unit time (for example, information related to a delivery amount expressed in the unit “milliliter/second”). The fluid delivery information 94C includes air supply amount information 94C1 and water supply amount information 94C2. The air supply amount information 94C1 is information related to the gas delivery amount (for example, information related to the amount of gas delivered per unit time). The water supply amount information 94C2 is information related to the liquid delivery amount (for example, information related to the amount of liquid delivered per unit time). The fluid delivery information 94C is an example of “fluid delivery information” according to the technology of the present disclosure.

The endoscope information 94 may include operation information of the endoscope 12 (for example, information based on a result of measurement of the operation time of the camera 48, and the like), information obtained from various sensors (for example, a pressure sensor and the like) incorporated in the tip portion 46, and/or the like.

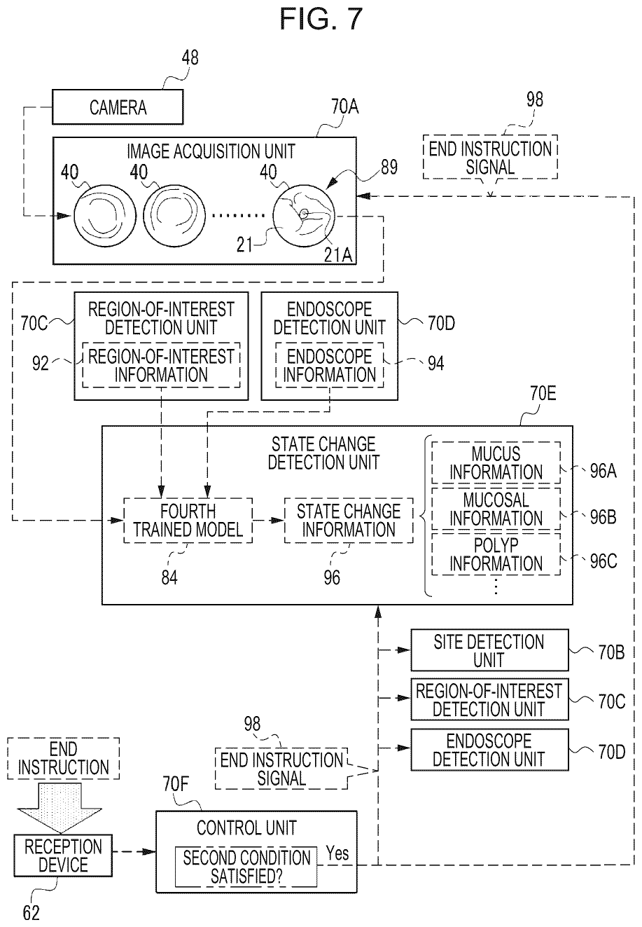

As an example, as illustrated in FIG. 7, the state change detection unit 70E performs the image recognition processing using the fourth trained model 84 on the time-series image group 89 to detect a state change of the observation target region 21. Examples of the state change of the observation target region 21 include a change in adhesive color, a change in mucosal state including a mucosal structure, and/or a change in mucus adhesion state.

The fourth trained model 84 is a trained model for AI-based object detection, and is optimized through machine learning performed for a neural network using fourth labeled data. Examples of the fourth labeled data include labeled data in which a plurality of images obtained in a time series by imaging the inside of the body using the camera 48, the region-of-interest information 92, and the endoscope information 94 are set as example data and state change information 96 related to a state change of the observation target region 21 is set as ground-truth data. While an example configuration is presented in which only one fourth trained model 84 is used by the state change detection unit 70E, this is merely an example. For example, the state change detection unit 70E may use a fourth trained model 84 selected from among a plurality of fourth trained models 84. In this case, desirably, the fourth trained models 84 are created through machine learning specific to the respective types of endoscopic examination, and the fourth trained model 84 corresponding to the type of the endoscopic examination currently being performed is selected and used by the state change detection unit 70E.

The state change of the observation target region 21 also includes a change in the region of interest 21A (for example, a change in the region of interest 21A caused by the delivery of the fluid 56). Examples of the change in the region of interest 21A include a change from a state in which mucus adheres to the region of interest 21A to a state in which a non-neoplastic polyp appears in the region of interest 21A. The state change information 96 includes mucus information 96A, mucosal information 96B, polyp information 96C, and the like. The mucus information 96A is information related to a change in mucus. Examples of the mucus information 96A include information indicating a change in the adhesion state of mucus and/or information indicating a change in the color of mucus. The mucosal information 96B is information related to a change in mucosal state. Examples of the mucosal information 96B include information indicating a change in mucosal structure, and/or information indicating a change in mucosal color. The polyp information 96C is information related to a polyp. Examples of the polyp information 96C include information indicating a change from a state in which mucus adheres to the region of interest 21A to a state in which a polyp (for example, a non-neoplastic polyp or a neoplastic polyp) appears in the region of interest 21A.

The state change detection unit 70E acquires the time-series image group 89 and inputs the acquired time-series image group 89 to the fourth trained model 84. The state change detection unit 70E further inputs the region-of-interest information 92 acquired by the region-of-interest detection unit 70C to the fourth trained model 84. The state change detection unit 70E further inputs the endoscope information 94 acquired by the endoscope detection unit 70D to the fourth trained model 84. When the plurality of time-series endoscopic images 40, the region-of-interest information 92, and the endoscope information 94 are input to the fourth trained model 84 in the manner described above, the fourth trained model 84 outputs state change information 96 corresponding to the plurality of time-series endoscopic images 40, the region-of-interest information 92, and the endoscope information 94 that are input. The state change detection unit 70E acquires the state change information 96 output from the fourth trained model 84. The state change information 96 acquired by the state change detection unit 70E is information related to a state change of the observation target region 21 currently being observed using the endoscope 12.

That is, the state change detection unit 70E performs AI image recognition processing based on the region-of-interest information 92 and the endoscope information 94 to detect a state change. The region-of-interest information 92 is information obtained through image recognition processing performed using the second trained model 80 selected based on the site information 90 (see FIG. 5). Thus, it can also be said that the detection of a state change by the state change detection unit 70E is indirectly implemented by performing the AI image recognition processing based on the site information 90.

When a second condition is satisfied, the control unit 70F outputs an end instruction signal 98 to the state change detection unit 70E. Examples of the second condition include conditions including a condition in which an end instruction is given to the endoscope 12 (in the example illustrated in FIG. 7, a condition in which the end instruction is received by the reception device 62). The end instruction refers to an instruction to end the AI image recognition processing.

When the end instruction signal 98 is input from the control unit 70F, the state change detection unit 70E ends the AI image recognition processing (in the example illustrated in FIG. 7, the image recognition processing using the fourth trained model 84) and deletes the state change information 96. The state change information 96 is an example of “first information that is information based on the image recognition processing” according to the technology of the present disclosure.

When the second condition is satisfied, the control unit 70F outputs the end instruction signal 98 also to the site detection unit 70B, the region-of-interest detection unit 70C, and the endoscope detection unit 70D. When the end instruction signal 98 is input from the control unit 70F, the site detection unit 70B ends the AI image recognition processing (in the example illustrated in FIG. 5, the image recognition processing using the first trained model 78) and deletes the site information 90 (see FIG. 5). When the end instruction signal 98 is input from the control unit 70F, the region-of-interest detection unit 70C ends the AI image recognition processing (in the example illustrated in FIG. 5, the image recognition processing using the second trained model 80) and deletes the region-of-interest information 92 (see FIG. 5). When the end instruction signal 98 is input from the control unit 70F, the endoscope detection unit 70D ends the AI image recognition processing (in the example illustrated in FIG. 6, the image recognition processing using the third trained model 82) and deletes the endoscope information 94 (see FIG. 6).

As described above, the information based on the AI image recognition processing (i.e., the site information 90, the region-of-interest information 92, the endoscope information 94, and the state change information 96) held by the processor 70 during a period from when the processor 70 starts the AI image recognition processing to when the processor 70 ends the AI image recognition processing is deleted when the AI image recognition processing ends (here, when the second condition is satisfied, as an example).

When the second condition is satisfied, the control unit 70F outputs the end instruction signal 98 also to the image acquisition unit 70A. When the end instruction signal 98 is input from the control unit 70F, the image acquisition unit 70A deletes the time-series image group 89 held at the current point in time. That is, the information used in the AI image recognition processing (i.e., the time-series image group 89) is also deleted when the AI image recognition processing ends (here, when the second condition is satisfied, as an example).

The image recognition processing performed using the first trained model 78 (see FIG. 5), the image recognition processing performed using the second trained model 80 (see FIG. 5), the image recognition processing performed using the third trained model 82 (see FIG. 6), and the image recognition processing performed using the fourth trained model 84 (see FIG. 7) are examples of “image recognition processing” according to the technology of the present disclosure.

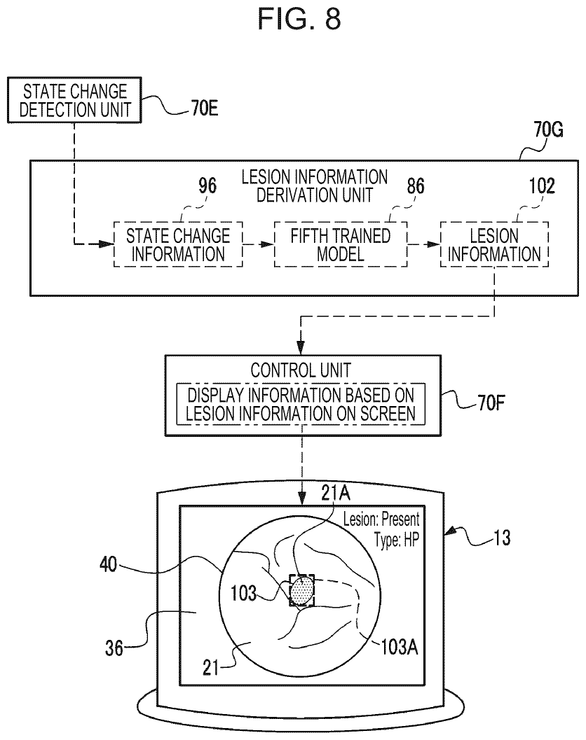

As an example, as illustrated in FIG. 8, the lesion information derivation unit 70G derives lesion information 102 related to a lesion in the observation target region 21, based on the state change detected by the state change detection unit 70E. Examples of the lesion information 102 include information indicating the presence or absence of a lesion and/or information indicating the type of the lesion. In this case, for example, the lesion information derivation unit 70G acquires the state change information 96 from the state change detection unit 70E, and performs AI derivation processing using the acquired state change information 96 to derive the lesion information 102. That is, the lesion information derivation unit 70G derives the lesion information 102 by performing AI derivation processing using the fifth trained model 86 on the state change information 96.

The fifth trained model 86 is optimized through machine learning performed for a neural network using fifth labeled data. Examples of the fifth labeled data include labeled data in which the state change information 96 is set as example data and the lesion information 102 is set as ground-truth data.

The lesion information derivation unit 70G acquires the state change information 96 from the state change detection unit 70E, and inputs the acquired state change information 96 to the fifth trained model 86. Accordingly, the fifth trained model 86 outputs lesion information 102 corresponding to the input state change information 96. The lesion information derivation unit 70G acquires the lesion information 102 output from the fifth trained model 86. The lesion information 102 acquired by the lesion information derivation unit 70G is information related to a lesion in the observation target region 21 currently being observed using the endoscope 12 (for example, a lesion in the region of interest 21A included in the observation target region 21).

The control unit 70F outputs information based on the lesion information 102 derived by the lesion information derivation unit 70G. The output destination is the display device 13. That is, the control unit 70F displays, on the screen 36 of the display device 13, the information based on the lesion information 102 derived by the lesion information derivation unit 70G. A first example of the information based on the lesion information 102 is information indicating the presence or absence of a lesion and information indicating the type of the lesion. A second example of the information based on the lesion information 102 is information that derives from the lesion information 102 (for example, information indicating the reliability of the lesion information 102). In the example illustrated in FIG. 8, the information based on the lesion information 102 is displayed at a position adjacent to the endoscopic image 40 on the screen 36. In the example illustrated in FIG. 8, furthermore, a detection frame 103A that surrounds an image region 103 representing the region of interest 21A depicted in the endoscopic image 40 is also displayed on the screen 36. For example, the detection frame 103A is a frame formed based on a bounding box used in the AI image recognition processing performed by the region-of-interest detection unit 70C. The display device 13 is an example of an “output destination” and a “display device” according to the technology of the present disclosure, and the information based on the lesion information 102 is an example of “information based on the image recognition processing” and “second information” according to the technology of the present disclosure.

While the display device 13 is illustrated as an example of the output destination of the information based on the lesion information 102, the technology of the present disclosure is not limited to this example. The output destination of the information based on the lesion information 102 may be, for example, an information processing apparatus such as a server to which the endoscope system 10 is communicably connected. The information based on the lesion information 102 may be stored in a storage medium (such as the NVM 74 and/or a memory of a device external to the endoscope 12). The information based on the lesion information 102 may be registered in an electronic medical record.

Next, the operation of a portion of the endoscope system 10 according to the technology of the present disclosure will be described with reference to FIGS. 9A and 9B.

FIGS. 9A and 9B illustrate an example of the flow of a medical support process performed by the processor 70. The flow of the medical support process illustrated in FIGS. 9A and 9B is an example of an “image processing method” according to the technology of the present disclosure.

In the medical support process illustrated in FIG. 9A, first, in step ST10, the image acquisition unit 70A determines whether imaging of one frame is performed by the camera 48. If imaging of one frame is not performed by the camera 48 in step ST10, the determination is negative, and the determination of step ST10 is performed again. If imaging of one frame is performed by the camera 48 in step ST10, the determination is affirmative, and the medical support process proceeds to step ST12.

In step ST12, the image acquisition unit 70A acquires an endoscopic image 40 for one frame from the camera 48. After the processing of step ST12 is performed, the medical support process proceeds to step ST14.

In step ST14, the image acquisition unit 70A determines whether endoscopic images 40 for a certain number of frames are held. If the endoscopic images 40 for the certain number of frames are not held in step ST14, the determination is negative, and the medical support process proceeds to step ST10. If the endoscopic images 40 for the certain number of frames are held in step ST14, the determination is affirmative, and the medical support process proceeds to step ST16.

In step ST16, the image acquisition unit 70A adds the endoscopic image 40 acquired in step ST12 to the time-series image group 89 in a FIFO manner to update the time-series image group 89. After the processing of step ST16 is performed, the medical support process proceeds to step ST18.

In step ST18, the control unit 70F determines whether the first condition is satisfied. If the first condition is not satisfied in step ST18, the determination is negative, and the medical support process proceeds to step ST10. If the first condition is satisfied in step ST18, the determination is affirmative, and the medical support process proceeds to step ST20.

In step ST20, the site detection unit 70B performs the image recognition processing using the first trained model 78 on the time-series image group 89 held by the image acquisition unit 70A to acquire the site information 90. After the processing of step ST20 is performed, the medical support process proceeds to step ST22.

In step ST22, the region-of-interest detection unit 70C selects, from among the plurality of second trained models 80, a second trained model 80 corresponding to the site information 90 acquired in step ST20. After the processing of step ST22 is performed, the medical support process proceeds to step ST24.

In step ST24, the region-of-interest detection unit 70C performs the image recognition processing using the second trained model selected in step ST22, on the time-series image group 89 held by the image acquisition unit 70A to acquire the region-of-interest information 92. After the processing of step ST24 is performed, the medical support process proceeds to step ST26 illustrated in FIG. 9B.

In step ST26, the endoscope detection unit 70D performs the image recognition processing using the third trained model 82 on the time-series image group 89 held by the image acquisition unit 70A to acquire the endoscope information 94. After the processing of step ST26 is performed, the medical support process proceeds to step ST28.

In step ST28, the state change detection unit 70E inputs the time-series image group 89 held by the image acquisition unit 70A, the region-of-interest information 92 acquired in step ST24, and the endoscope information 94 acquired in step ST26 to the fourth trained model 84 to acquire the state change information 96 from the fourth trained model 84. After the processing of step ST28 is performed, the medical support process proceeds to step ST30.

In step ST30, the control unit 70F determines whether the second condition is satisfied. If the second condition is not satisfied in step ST30, the determination is negative, and the medical support process proceeds to step ST34. If the second condition is satisfied in step ST30, the determination is affirmative, and the medical support process proceeds to step ST32.

In step ST32, the processor 70 ends the AI image recognition processing and deletes the information based on the image recognition processing, and the like. That is, the site detection unit 70B ends the AI image recognition processing (in the example illustrated in FIG. 5, the image recognition processing using the first trained model 78) and deletes the site information 90 (see FIG. 5). The region-of-interest detection unit 70C ends the AI image recognition processing (in the example illustrated in FIG. 5, the image recognition processing using the second trained model 80) and deletes the region-of-interest information 92 (see FIG. 5). The endoscope detection unit 70D ends the AI image recognition processing (in the example illustrated in FIG. 6, the image recognition processing using the third trained model 82) and deletes the endoscope information 94 (see FIG. 6). The image acquisition unit 70A deletes the time-series image group 89 held at the current point in time. After the processing of step ST32 is performed, the medical support process proceeds to step ST34.

In step ST34, the lesion information derivation unit 70G inputs the state change information 96 acquired in step ST28 to the fifth trained model 86 to derive the lesion information 102 corresponding to the state change information 96 from the fifth trained model 86. After the processing of step ST34 is performed, the medical support process proceeds to step ST36.

In step ST36, the control unit 70F displays information based on the lesion information 102 derived in step ST34 on the screen 36 of the display device 13. After the processing of step ST36 is performed, the medical support process proceeds to step ST38.

In step ST38, the control unit 70F determines whether a condition for ending the medical support process is satisfied. Examples of the condition for ending the medical support process include a condition in which an instruction to end the medical support process is given to the endoscope system 10 (for example, a condition in which the instruction to end the medical support process is received by the reception device 62).

If the condition for ending the medical support process is not satisfied in step ST38, the determination is negative, and the medical support process proceeds to step ST10 illustrated in FIG. 9A. If the condition for ending the medical support process is satisfied in step ST38, the determination is affirmative, and the medical support process ends.

As described above, in the endoscope system 10, the AI image recognition processing is performed on the time-series image group 89 generated by the camera 48 (for example, a plurality of time-series endoscopic images 40 that can identify the condition of the observation target region 21 before and after the delivery of the fluid 56) to detect a state change of the observation target region 21. This enables more accurate detection of a state change of the observation target region 21 than detection of a state change of the observation target region 21 using only a single endoscopic image 40 (for example, only a single endoscopic image 40 obtained at a timing at which the fluid 56 is not delivered). As described above, accurate detection of a state change of the observation target region 21 enables, for example, the doctor 14 to accurately differentiate a sessile serrated lesion, Barrett's esophagus-associated cancer, or the like, which is generally known as a lesion difficult to differentiate.

In the endoscope system 10, furthermore, the state change detection unit 70E detects a state change of the observation target region 21, namely, a change in adhesive color, a change in mucosal state including a mucosal structure, and/or a change in mucus adhesion state. Accordingly, a change in adhesive color, a change in mucosal state including a mucosal structure, and/or a change in mucus adhesion state can be detected with higher accuracy as a state change of the observation target region 21 than the detection of a change in adhesive color, a change in mucosal state including a mucosal structure, and/or a change in mucus adhesion state using only a single endoscopic image 40.

In the endoscope system 10, furthermore, the endoscope detection unit 70D performs AI image recognition processing on the time-series image group 89 to acquire the endoscope information 94. Then, the state change detection unit 70E detects a state change based on the endoscope information 94. This enables more accurate detection of a state change than the detection of a state change without consideration of the endoscope information 94. The endoscope information 94 includes the fluid delivery information 94C. This enables more accurate detection of a state change than the detection of a state change without consideration of the fluid delivery information 94C.

In the endoscope system 10, furthermore, the AI image recognition processing is started when the first condition is satisfied. Thus, the AI image recognition processing can be started at a more suitable timing than in a case where the AI image recognition processing is unconditionally started. In this embodiment, a condition in which a start instruction is given to the endoscope 12 (in the example illustrated in FIG. 5, a condition in which the start instruction is received by the reception device 62) is applied as the first condition. This enables the AI image recognition processing to be started at a timing intended by the doctor 14.

In the endoscope system 10, furthermore, the AI image recognition processing ends when the second condition is satisfied. Thus, the AI image recognition processing can be ended at a more suitable timing than in a case where the AI image recognition processing is unconditionally ended. In this embodiment, a condition including a condition in which an end instruction is given to the endoscope 12 (in the example illustrated in FIG. 7, a condition in which the end instruction is received by the reception device 62) is applied as the second condition. This enables the AI image recognition processing to be ended at a timing intended by the doctor 14.

In the endoscope system 10, furthermore, the information based on the AI image recognition processing is deleted when the second condition is satisfied. Thus, the information based on the AI image recognition processing can be prevented from being deleted at a timing unintended by the doctor 14, as compared to a case where the information based on the AI image recognition processing is unconditionally deleted.

In the endoscope system 10, furthermore, the information based on the AI image recognition processing is held during a period from the start to the end of the AI image recognition processing. When the AI image recognition processing ends, the information based on the AI image recognition processing is deleted. Accordingly, a process using the information based on the AI image recognition processing (for example, a process performed by the lesion information derivation unit 70G) can be performed until the end of the AI image recognition processing, and the information based on the AI image recognition processing can be deleted at a timing at which the AI image recognition processing is no longer necessary.

In the endoscope system 10, furthermore, the state change detection unit 70E detects a state change of the observation target region 21 on condition that the region-of-interest information 92 is acquired by the region-of-interest detection unit 70C. This enables detection of a state change of the observation target region 21 at a more suitable timing (for example, a timing at which the region of interest 21A is included in the observation target region 21) than detection of a state change of the observation target region 21 at a stage preceding the detection of the region-of-interest information 92. In other words, for example, it is possible to prevent a state change of the observation target region 21 from being detected in a case where the region of interest 21A is not included in the observation target region 21.

In the endoscope system 10, furthermore, the state change detection unit 70E detects a state change of the observation target region 21 on condition that the site information 90 is acquired by the site detection unit 70B. This enables detection of a state change of the observation target region 21 at a more suitable timing (for example, a timing at which a designated site is depicted in the endoscopic image 40 as a site corresponding to the observation target region 21) than detection of a state change of the observation target region 21 at a stage preceding the detection of the site information 90. In other words, for example, it is possible to prevent a state change of the observation target region 21 from being detected in a case where the site depicted in the endoscopic image 40 is a site different from the designated site.

In the endoscope system 10, furthermore, the lesion information derivation unit 70G derives the lesion information 102 based on the state change detected by the state change detection unit 70E. This enables derivation of more reliable lesion information 102 than in a case where the state change of the observation target region 21 is predicted using only a single endoscopic image 40 and the lesion information 102 is derived based on the predicted state change.

In the endoscope system 10, furthermore, the state change detection unit 70E detects a change in the region of interest 21A. For example, the state change detection unit 70E detects, as the change in the region of interest 21A, a change from a state in which mucus adheres to the region of interest 21A to a state in which a non-neoplastic polyp appears in the region of interest 21A. Accordingly, it is possible to derive more reliable lesion information 102 than in a case where, as a change in the region of interest 21A, a change from a state in which mucus adheres to the region of interest 21A to a state in which a non-neoplastic polyp appears in the region of interest 21A is predicted using only a single endoscopic image 40 and the lesion information 102 is derived based on the predicted change.

In the endoscope system 10, furthermore, the control unit 70F outputs the information based on the lesion information 102. Examples of the output destination include the display device 13 and/or an information processing apparatus (a server, a personal computer, or a tablet terminal). This enables a process using the information based on the lesion information 102 to be performed at the output destination of the information based on the lesion information 102.

In the endoscope system 10, furthermore, the control unit 70F displays, on the screen 36 of the display device 13, the information based on the lesion information 102. This allows the user to grasp the information based on the lesion information 102.

While the embodiment described above provides an example configuration in which the time-series image group 89, the region-of-interest information 92, and the endoscope information 94 are input to the fourth trained model 84 and the state change information 96 corresponding thereto is output from the fourth trained model 84, the technology of the present disclosure is not limited to this configuration. For example, the site information 90 may further be input to the fourth trained model 84, and the state change information 96 corresponding to the site information 90, the time-series image group 89, the region-of-interest information 92, and the endoscope information 94 may be output from the fourth trained model 84. In this case, desirably, as the fourth labeled data used to create the fourth trained model 84, labeled data may be used in which, for example, a plurality of images obtained in a time series by imaging the inside of the body using the camera 48, the site information 90, the region-of-interest information 92, and the endoscope information 94 are set as example data and the state change information 96 related to a state change of the observation target region 21 is set as ground-truth data.

In addition to the time-series image group 89, the site information 90, the region-of-interest information 92, and/or the endoscope information 94 may be input to the fourth trained model 84, and the state change information 96 corresponding to the input may be output from the fourth trained model 84. Also in this case, desirably, as the fourth labeled data used to create the fourth trained model 84, labeled data may be used in which the information input to the fourth trained model 84 is set as example data, as described above. The fourth labeled data used to create the fourth trained model 84 may be labeled data in which a plurality of images obtained in a time series by imaging the inside of the body using the camera 48 are set as example data and the state change information 96 related to a state change of the observation target region 21 is set as ground-truth data. In this case, when the time-series image group 89 is input to the fourth trained model 84, the state change information 96 corresponding to the input time-series image group 89 is output from the fourth trained model 84. That is, the state change detection unit 70E performs the AI image recognition processing (i.e., the image recognition processing using the fourth trained model 84) on the time-series image group 89 to detect a state change of the observation target region 21.

While the embodiment described above provides an example configuration in which the AI image recognition processing is performed based on a start instruction given to the endoscope 12, the technology of the present disclosure is not limited to this configuration. For example, the AI image recognition processing may be performed based on the operation of the endoscope 12. In this case, for example, the operation of the endoscope 12 is identified from the endoscope information 94.

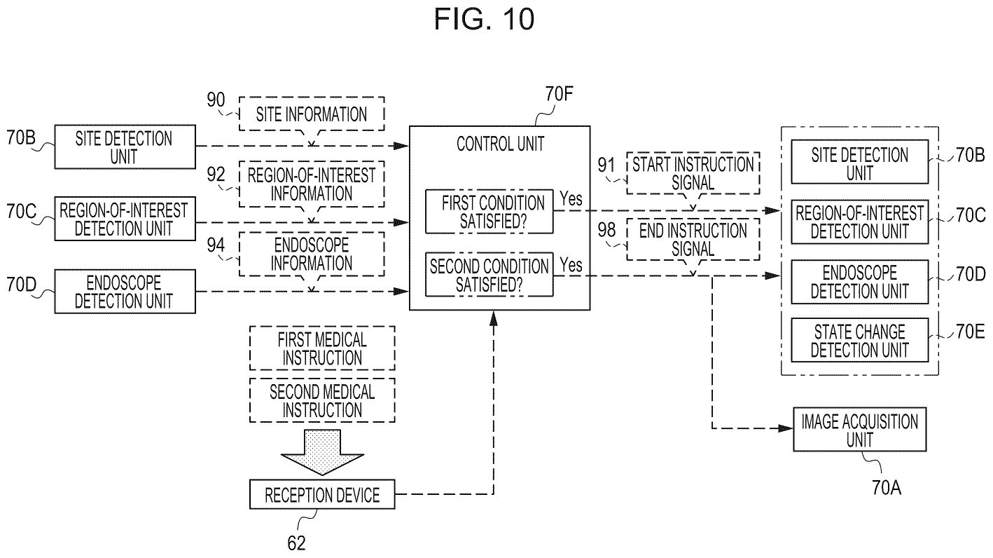

To perform the AI image recognition processing based on the operation of the endoscope 12, as an example, as illustrated in FIG. 10, the control unit 70F outputs the start instruction signal 91 to the site detection unit 70B, the region-of-interest detection unit 70C, the endoscope detection unit 70D, and the state change detection unit 70E when a first condition using the endoscope information 94 is satisfied, for example. A first example of the first condition using the endoscope information 94 is a condition in which the tip portion 46 of the endoscope 12 has stopped moving. Whether the tip portion 46 of the endoscope 12 has stopped moving is determined based on the operating speed information 94B. A second example of the first condition using the endoscope information 94 is a condition in which the moving speed of the tip portion 46 of the endoscope 12 has decreased. Whether the moving speed of the tip portion 46 of the endoscope 12 has decreased is determined based on the operating speed information 94B. The decrease in the moving speed refers to, for example, a decrease in the moving speed to less than a specified speed (for example, a decrease from several tens of millimeters/second to several millimeters/second).

Accordingly, since the AI image recognition processing is performed based on the operation of the endoscope 12, the AI image recognition processing can be performed at a more suitable timing than in a case where the AI image recognition processing is performed regardless of the operation of the endoscope 12. Further, the AI image recognition processing is started when a condition in which the tip portion 46 of the endoscope 12 has stopped moving or a condition in which the moving speed of the tip portion 46 of the endoscope 12 has decreased is satisfied. Thus, the AI image recognition processing can be started at a more suitable timing than in a case where the AI image recognition processing is started regardless of the moving speed of the tip portion 46 of the endoscope 12.

Alternatively, the AI image recognition processing may be performed based on the region-of-interest information 92. In this case, for example, as illustrated in FIG. 10, when a first condition using the region-of-interest information 92 is satisfied, the control unit 70F outputs the start instruction signal 91 to the site detection unit 70B, the region-of-interest detection unit 70C, the endoscope detection unit 70D, and the state change detection unit 70E. A first example of the first condition using the region-of-interest information 92 is a condition in which the region of interest 21A is included in the observation target region 21. Whether the region of interest 21A is included in the observation target region 21 is determined based on the region-of-interest information 92. A second example of the first condition using the region-of-interest information 92 is a condition in which a specific region of interest 21A (for example, a region to which mucus adheres) is included in the observation target region 21. Whether the specific region of interest 21A is included in the observation target region 21 is determined based on the region-of-interest information 92.

Accordingly, since the AI image recognition processing is performed based on the region-of-interest information 92, the AI image recognition processing can be performed at a more suitable timing than in a case where the AI image recognition processing is performed regardless of the region-of-interest information 92. Further, the AI image recognition processing is started when a condition in which the region of interest 21a is included in the observation target region 21 is satisfied. Thus, the AI image recognition processing can be started at a more suitable timing than in a case where the AI image recognition processing is started regardless of whether the region of interest 21a is included in the observation target region 21. In other words, for example, it is possible to prevent the image recognition processing from being started in a case where the region of interest 21A is not included in the observation target region 21.

Alternatively, the AI image recognition processing may be performed based on the site information 90. In this case, for example, as illustrated in FIG. 10, when a first condition using the site information 90 is satisfied, the control unit 70F outputs the start instruction signal 91 to the site detection unit 70B, the region-of-interest detection unit 70C, the endoscope detection unit 70D, and the state change detection unit 70E. Examples of the first condition using the site information 90 include a condition in which the site corresponding to the observation target region 21 is a site designated as an observation target (for example, the ascending colon). The site may be designated via, for example, the reception device 62 or a communication device capable of communicating with the endoscope system 10. The site may be designated by any method. Whether the site corresponding to the observation target region 21 is a site designated as an observation target is determined based on the site information 90.

Accordingly, since the AI image recognition processing is performed based on the site information 90, the AI image recognition processing can be performed at a more suitable timing than in a case where the AI image recognition processing is performed regardless of the site information 90. Further, the AI image recognition processing is started when a condition in which the site corresponding to the observation target region 21 is a site designated as an observation target is satisfied. Thus, the AI image recognition processing can be started at a more suitable timing than in a case where the AI image recognition processing is started regardless of the site corresponding to the observation target region 21. In other words, for example, it is possible to prevent the image recognition processing from being started in a case where the site corresponding to the observation target region 21 is a site (for example, the descending colon) different from a site (for example, the ascending colon) designated as an observation target.

Alternatively, the AI image recognition processing and the detection of a state change by the state change detection unit 70E may be performed based on a first medical instruction given to the endoscope 12. In this case, for example, as illustrated in FIG. 10, when a first condition using the first medical instruction is satisfied, the control unit 70F outputs the start instruction signal 91 to the site detection unit 70B, the region-of-interest detection unit 70C, the endoscope detection unit 70D, and the state change detection unit 70E. The first medical instruction is an example of a “medical instruction” according to the technology of the present disclosure. The first medical instruction refers to, for example, an instruction given to the endoscope 12 via the operation section 42 and/or the reception device 62. Examples of the first medical instruction include an instruction to deliver the fluid 56 from the treatment opening 52. Accordingly, since the image recognition processing is performed based on the first medical instruction given to the endoscope 12, the AI image recognition processing can be performed at a more suitable timing than in a case where the AI image recognition processing is performed regardless of the presence or absence of the first medical instruction for the endoscope 12. Further, a state change can be detected at a more suitable timing than in a case where the state change detection unit 70E detects a state change regardless of the presence or absence of the first medical instruction given to the endoscope 12.

Alternatively, the control unit 70F may output the start instruction signal 91 when a plurality of first conditions designated in advance are satisfied among the first condition using the start instruction, the first condition using the endoscope information 94, the first condition using the region-of-interest information 92, the first condition using the site information 90, and the first condition using the first medical instruction described in the embodiment described above.

While the embodiment described above provides an example configuration in which the AI image recognition processing ends based on an end instruction given to the endoscope 12, the technology of the present disclosure is not limited to this configuration. For example, the AI image recognition processing may end based on the operation of the endoscope 12. In this case, for example, the operation of the endoscope 12 is identified from the endoscope information 94.

To end the AI image recognition processing based on the operation of the endoscope 12, as an example, as illustrated in FIG. 10, the control unit 70F outputs the end instruction signal 98 to the site detection unit 70B, the region-of-interest detection unit 70C, the endoscope detection unit 70D, and the state change detection unit 70E when a second condition using the endoscope information 94 is satisfied, for example. A first example of the second condition using the endoscope information 94 is a condition in which the tip portion 46 of the endoscope 12 has started moving. Whether the tip portion 46 of the endoscope 12 has started moving is determined based on the operating speed information 94B. A second example of the first condition using the endoscope information 94 is a condition in which the moving speed of the tip portion 46 of the endoscope 12 has increased. Whether the moving speed of the tip portion 46 of the endoscope 12 has increased is determined based on the operating speed information 94B. The increase in the moving speed refers to, for example, an increase in the moving speed to the specified speed or higher (for example, an increase from several millimeters/second to several tens of millimeters/second).

Accordingly, since the AI image recognition processing ends based on the operation of the endoscope 12, the AI image recognition processing can be ended at a more suitable timing than in a case where the AI image recognition processing ends regardless of the operation of the endoscope 12. Further, the AI image recognition processing ends when a condition in which the tip portion 46 of the endoscope 12 has started moving or a condition in which the moving speed of the tip portion 46 of the endoscope 12 has increased is satisfied. Thus, the AI image recognition processing can be ended at a more suitable timing than in a case where the AI image recognition processing is ended regardless of the moving speed of the tip portion 46 of the endoscope 12.

Alternatively, the AI image recognition processing may end based on the region-of-interest information 92. In this case, for example, as illustrated in FIG. 10, when a second condition using the region-of-interest information 92 is satisfied, the control unit 70F outputs the end instruction signal 98 to the site detection unit 70B, the region-of-interest detection unit 70C, the endoscope detection unit 70D, and the state change detection unit 70E. A first example of the second condition using the region-of-interest information 92 is a condition in which the region of interest 21A is not included in the observation target region 21. Whether the region of interest 21A is not included in the observation target region 21 is determined based on the region-of-interest information 92. A second example of the second condition using the region-of-interest information 92 is a condition in which a specific region of interest 21A (for example, a region to which mucus adheres) is not included in the observation target region 21. Whether the specific region of interest 21A is not included in the observation target region 21 is determined based on the region-of-interest information 92.

Accordingly, since the AI image recognition processing ends based on the region-of-interest information 92, the AI image recognition processing can be ended at a more suitable timing than in a case where the AI image recognition processing ends regardless of the region-of-interest information 92. Further, the AI image recognition processing ends when a condition in which the region of interest 21a is not included in the observation target region 21 is satisfied. Thus, the AI image recognition processing can be ended at a more suitable timing than in a case where the AI image recognition processing ends regardless of whether the region of interest 21a is included in the observation target region 21. In other words, for example, it is possible to prevent the image recognition processing from being ended in a case where the region of interest 21A is included in the observation target region 21.

Alternatively, the AI image recognition processing may end based on the site information 90. In this case, for example, as illustrated in FIG. 10, when a second condition using the site information 90 is satisfied, the control unit 70F outputs the end instruction signal 98 to the site detection unit 70B, the region-of-interest detection unit 70C, the endoscope detection unit 70D, and the state change detection unit 70E. Examples of the second condition using the site information 90 include a condition in which the site corresponding to the observation target region 21 is a site (for example, the descending colon) different from a site (for example, the ascending colon) designated as an observation target. Whether the site corresponding to the observation target region 21 is a site different from a site designated as an observation target is determined based on the site information 90.

Accordingly, since the AI image recognition processing ends based on the site information 90, the AI image recognition processing can be ended at a more suitable timing than in a case where the AI image recognition processing ends regardless of the site information 90. Further, the AI image recognition processing ends when a condition in which the site corresponding to the observation target region 21 is a site different from a site designated as an observation target is satisfied. Thus, the AI image recognition processing can be ended at a more suitable timing than in a case where the AI image recognition processing ends regardless of the site corresponding to the observation target region 21. In other words, for example, it is possible to prevent the image recognition processing from being ended in a case where the site corresponding to the observation target region 21 is a site (for example, the ascending colon) designated as an observation target.

Alternatively, the AI image recognition processing may be performed based on a second medical instruction given to the endoscope 12. In this case, for example, as illustrated in FIG. 10, when a second condition using the second medical instruction is satisfied, the control unit 70F outputs the end instruction signal 98 to the site detection unit 70B, the region-of-interest detection unit 70C, the endoscope detection unit 70D, and the state change detection unit 70E. The second medical instruction refers to, for example, an instruction given to the endoscope 12 via the operation section 42 and/or the reception device 62. Examples of the second medical instruction include an instruction to stop the delivery of the fluid 56 from the treatment opening 52. Accordingly, since the image recognition processing is performed based on the second medical instruction given to the endoscope 12, the AI image recognition processing can be ended at a more suitable timing than in a case where the AI image recognition processing ends regardless of the presence or absence of the second medical instruction for the endoscope 12.

Alternatively, the control unit 70F may output the end instruction signal 98 when a plurality of second conditions designated in advance are satisfied among the second condition using the end instruction, the second condition using the endoscope information 94, the second condition using the region-of-interest information 92, the second condition using the site information 90, and the second condition using the second medical instruction described in the embodiment described above.

As an example, as illustrated in FIG. 10, the control unit 70F may output the end instruction signal 98 to the image acquisition unit 70A when one or more of the second conditions described above are satisfied. Also in this case, as in the embodiment described above, the image acquisition unit 70A deletes the time-series image group 89 held at the current point in time.

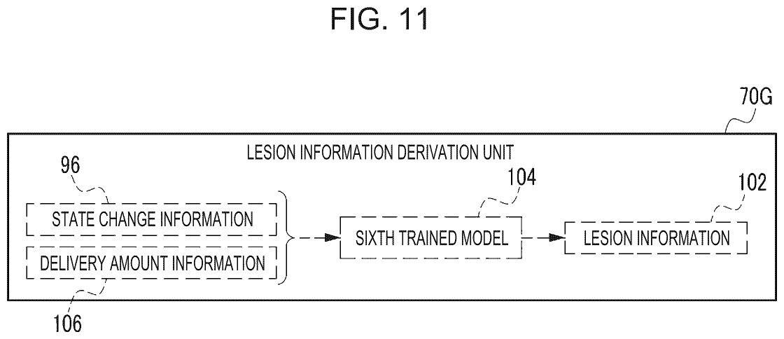

While the embodiment described above provides an example configuration in which the lesion information derivation unit 70G performs a process using the fifth trained model 86 to derive the lesion information 102, the technology of the present disclosure is not limited to this configuration. For example, as illustrated in FIG. 11, the lesion information derivation unit 70G may derive the lesion information 102 by performing a process using a sixth trained model 104.

The sixth trained model 104 is optimized through machine learning performed for a neural network using sixth labeled data. Examples of the sixth labeled data include labeled data in which the state change information 96 and delivery amount information 106 are set as example data and the lesion information 102 is set as ground-truth data. The delivery amount information 106 is an example of “delivery amount information” according to the technology of the present disclosure. The delivery amount information 106 is information indicating the delivery amount of the fluid 56 (see FIG. 2). For example, the delivery amount information 106 is included in the endoscope information 94. Examples of the delivery amount information 106 include the air supply amount information 94C1 and/or the water supply amount information 94C2. The delivery amount information 106 may be information obtained by measuring the delivery amount using a sensor or the like, or may be information obtained from a value received by the reception device 62.

The lesion information derivation unit 70G acquires the state change information 96 and the delivery amount information 106, and inputs the acquired state change information 96 and delivery amount information 106 to the sixth trained model 104. Accordingly, the sixth trained model 104 outputs lesion information 102 corresponding to the state change information 96 and the delivery amount information 106 that are input. The lesion information derivation unit 70G acquires the lesion information 102 output from the sixth trained model 86. The lesion information 102 acquired by the lesion information derivation unit 70G is information related to a lesion in the observation target region 21 currently being observed using the endoscope 12 (for example, a lesion in the region of interest 21A included in the observation target region 21).

In the example illustrated in FIG. 11, as described above, the lesion information 102 is derived based on the state change information 96 and the delivery amount information 106. This enables derivation of more reliable lesion information 102 than in a case where the lesion information 102 is derived without using the delivery amount information 106.

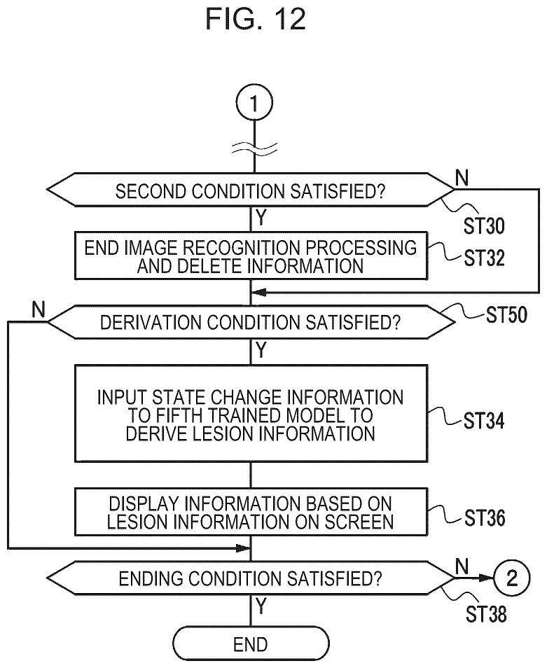

While the embodiment described above provides an example configuration in which the lesion information derivation unit 70G derives the lesion information 102 on condition that a state change of the observation target region 21 is detected by the state change detection unit 70E, the condition for deriving the lesion information 102 is not limited to this configuration. For example, as illustrated in FIG. 12, the processing of step ST50, which is processing for determining a condition for deriving the lesion information 102, may be inserted between the processing of step ST32 and the processing of step ST34 of the medical support process.

In step ST50 of the medical support process illustrated in FIG. 12, the lesion information derivation unit 70G determines whether a condition for deriving the lesion information 102 (hereinafter referred to as a “derivation condition”) is satisfied.

A first example of the derivation condition is a condition in which a timing determined based on the operation of the endoscope 12 is a first timing. The first timing refers to, for example, a timing at which the tip portion 46 of the endoscope 12 has started moving or a timing at which the moving speed of the tip portion 46 of the endoscope 12 has increased. For example, whether the timing determined based on the operation of the endoscope 12 is the first timing is determined based on the operating speed information 94B (see FIG. 6).

A second example of the derivation condition is a condition in which the second medical instruction described above is given to the endoscope 12. The second medical instruction is an example of a “medical instruction” according to the technology of the present disclosure.

A third example of the derivation condition is a condition in which the region-of-interest information 92 is information related to a specific region of interest 21A (for example, a region to which mucus adheres). The specific region of interest 21A is an example of a “specific region of interest” according to the technology of the present disclosure.

A fourth example of the derivation condition is a condition in which the site information 90 is information related to a specific site (for example, the ascending colon). The specific site is an example of a “specific site” according to the technology of the present disclosure.

If the derivation condition is not satisfied in step ST50, the determination is negative, and the medical support process illustrated in FIG. 12 proceeds to step ST38. If the derivation condition is satisfied in step ST50, the determination is affirmative, and the medical support process illustrated in FIG. 12 proceeds to step ST34. Step ST34 is performed in the way described in the embodiment described above to derive the lesion information 102.

In the example illustrated in FIG. 12, as described above, the lesion information 102 is derived when a condition in which the timing determined based on the operation of the endoscope 12 is the first timing is satisfied as a derivation condition. This enables more accurate derivation of the lesion information 102 for the observation target region 21 intended by the user than in a case where the lesion information 102 is derived without consideration of the operation of the endoscope 12.

In the example illustrated in FIG. 12, furthermore, the lesion information 102 is derived when a condition in which the second medical instruction described above is given to the endoscope 12 is satisfied as a derivation condition. This enables more accurate derivation of the lesion information 102 for the observation target region 21 intended by the user than in a case where the lesion information 102 is derived without consideration of the second medical instruction given to the endoscope 12.

In the example illustrated in FIG. 12, furthermore, the lesion information 102 is derived when a condition in which the region-of-interest information 92 is information related to a specific region of interest 21A is satisfied as a derivation condition. This enables more accurate derivation of the lesion information 102 for the specific region of interest 21A than in a case where the lesion information 102 is derived without consideration of the region-of-interest information 92.

In the example illustrated in FIG. 12, furthermore, the lesion information 102 is derived when a condition in which the site information 90 is information related to a specific site is satisfied as a derivation condition. This enables more accurate derivation of the lesion information 102 for a specific site than in a case where the lesion information 102 is derived without consideration of the site information 90.

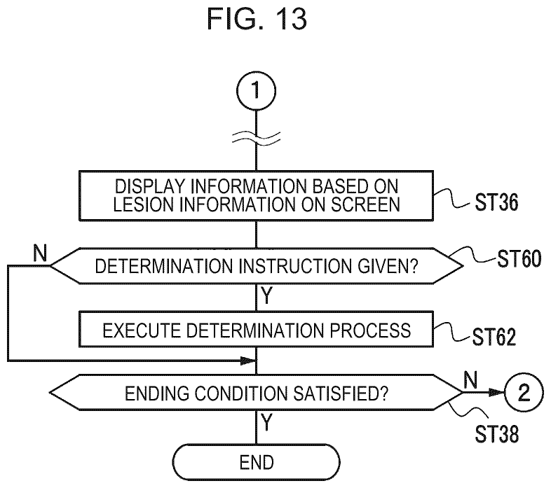

The lesion information 102 derived by the lesion information derivation unit 70G may be determined by the control unit 70F in accordance with a given instruction (for example, an instruction given from the doctor 14). In this case, for example, as illustrated in FIG. 13, the processing of step ST60 and the processing of step ST62 are inserted between the processing of step ST36 and the processing of step ST38 of the medical support process.

In step ST60 illustrated in FIG. 13, the control unit 70F determines whether an instruction to determine the lesion information 102 derived by the lesion information derivation unit 70G (hereinafter referred to as a “determination instruction”) is given to the endoscope 12 (for example, whether the determination instruction is received by the reception device 62). If the determination instruction is not given to the endoscope 12 in step ST60, the determination is negative, and the medical support process illustrated in FIG. 13 proceeds to step ST38. If the determination instruction is given to the endoscope 12 in step ST60, the determination is affirmative, and the medical support process illustrated in FIG. 13 proceeds to step ST62.

In step ST62, the control unit 70F executes a determination process. A first example of the determination process is a process of displaying determination information on the screen 36 of the display device 13. An example of the determination information is desirably a mark, text, or the like that allows the user to visually grasp that the lesion information 102 is determined. A second example of the determination process is a process of changing the display mode of the information based on the lesion information 102 displayed on the screen 36. In this case, the display modes before and after the change allow the user to visually grasp that the lesion information 102 is determined. A third example of the determination process is a process of causing an audio playback device to play back audio indicating that the lesion information 102 is determined. A fourth example of the determination process is a process of registering the lesion information 102 in an electronic medical record or the like in association with the current time (for example, the time at which the determination instruction is received). After the processing of step ST62 is performed, the medical support process illustrated in FIG. 13 proceeds to step ST38.

In the example illustrated in FIG. 13, as described above, the lesion information 102 derived by the lesion information derivation unit 70G is determined by the control unit 70F in accordance with the determination instruction. This allows the doctor 14 to confirm through the screen 36 that the lesion information 102 derived by the lesion information derivation unit 70G is not erroneous, and then determine the lesion information 102.

In the embodiment described above, the AI image recognition processing is used as an example. However, the technology of the present disclosure is not limited to this example, and non-AI image recognition processing (for example, image recognition processing using template matching) may be used instead of the AI image recognition processing. The AI image recognition processing and the non-AI image recognition processing may be used in combination.

While the embodiment described above provides an example configuration in which the lesion information 102 is derived by AI processing, the technology of the present disclosure is not limited to this configuration, and the lesion information 102 may be derived by non-AI processing. In this case, for example, the lesion information 102 is desirably derived using a table in which the state change information 96 and the lesion information 102 are associated with each other or using an arithmetic expression in which a value indicating the state change information 96 is the independent variable and a value indicating the lesion information 102 is the dependent variable.

While the embodiment described above provides an example configuration in which the medical support process is performed by the processor 70 of the computer 64 included in the endoscope 12, the technology of the present disclosure is not limited to this configuration, and a device external to the endoscope 12 may perform the medical support process. Examples of the device external to the endoscope 12 include at least one server and/or at least one personal computer communicably connected to the endoscope 12. Alternatively, the medical support process may be performed by a plurality of devices in a distributed manner.

While the embodiment described above provides an example configuration in which the medical support processing program 76 is stored in the NVM 74, the technology of the present disclosure is not limited to this configuration. For example, the medical support processing program 76 may be stored in a portable non-transitory storage medium such as an SSD or a USB memory. The medical support processing program 76 stored in the non-transitory storage medium is installed in the computer 64 of the endoscope 12. The processor 70 executes the medical support process in accordance with the medical support processing program 76.

Alternatively, the medical support processing program 76 may be stored in a storage device of another computer, a server, or the like connected to the endoscope 12 via a network, and the medical support processing program 76 may be downloaded in response to a request from the endoscope 12 and installed in the computer 64.

Not all, but a portion of the medical support processing program 76 may be stored in a storage device of another computer, a server device, or the like connected to the endoscope 12 or in the NVM 74.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards mentioned herein are incorporated herein by reference to the same extent as if each individual publication, patent application, and technical standard were specifically and individually indicated to be incorporated by reference.

What is claimed is:

1. An image processing apparatus comprising:
a processor,
the processor is configured to:
acquire a plurality of medical images along a time series, the plurality of medical images depicting an observation target region; and
perform image recognition processing on the plurality of medical images to detect a state change of the observation target region.

2. The image processing apparatus according to claim 1, wherein
the state change includes a change in adhesive color, a change in mucosal state including mucosal structure, and/or a change in mucus adhesion state.

3. The image processing apparatus according to claim 1, wherein
the processor is configured to start the image recognition processing in response to a first condition being satisfied.

4. The image processing apparatus according to claim 3, wherein
the first condition includes a condition in which an instruction to start the image recognition processing is given.

5. The image processing apparatus according to claim 3, wherein
the first condition includes a condition in which a region of interest is included in the observation target region.

6. The image processing apparatus according to claim 3, wherein
the first condition includes a condition in which a site corresponding to the observation target region is a site designated as an observation target.

7. The image processing apparatus according to claim 1, wherein
the processor is configured to end the image recognition processing in response to a second condition being satisfied.

8. The image processing apparatus according to claim 7, wherein
the processor is configured to delete first information that is information based on the image recognition processing, in response to the second condition being satisfied.

9. The image processing apparatus according to claim 8, wherein
the first information is held during a period from a start of the image recognition processing to an end of the image recognition processing, and
the processor is configured to delete the first information in response to the end of the image recognition processing.

10. The image processing apparatus according to claim 7, wherein
in a case where the plurality of medical images are generated by an endoscope,
the second condition includes a condition in which a tip portion of the endoscope has started moving or a condition in which a moving speed of the tip portion has increased.

11. The image processing apparatus according to claim 7, wherein
the second condition includes a condition in which an instruction to end the image recognition processing is given.

12. The image processing apparatus according to claim 7, wherein
the second condition includes a condition in which a region of interest is not included in the observation target region.

13. The image processing apparatus according to claim 1, wherein
in a case where the plurality of medical images are generated by an endoscope,
the processor is configured to detect the state change based on an operation of the endoscope.

14. The image processing apparatus according to claim 1, wherein
the processor is configured to:
acquire region-of-interest information related to a region of interest included in the observation target region; and
detect the state change on condition that the region-of-interest information is acquired.

15. The image processing apparatus according to claim 1, wherein
the processor is configured to:
acquire site information related to a site corresponding to the observation target region;
detect the state change on condition that the site information is acquired.

16. The image processing apparatus according to claim 1, wherein
the processor is configured to derive lesion information related to a lesion in the observation target region, based on the state change.

17. The image processing apparatus according to claim 16, wherein
the observation target region includes a region of interest,
the state change includes a change in the region of interest, and
the change in the region of interest is a change from a state in which mucus adheres to the region of interest to a state in which a non-neoplastic polyp appears in the region of interest.

18. The image processing apparatus according to claim 16, wherein
the observation target region includes a region of interest, and
in a case where the plurality of medical images are generated by an endoscope and a fluid is delivered from the endoscope into a body including the observation target region,
the state change includes a change in the region of interest caused by a delivery of the fluid, and
the processor is configured to:
acquire delivery amount information indicating a delivery amount of the fluid; and
derive the lesion information based on the state change and the delivery amount information.

19. The image processing apparatus according to claim 18, wherein
the change in the region of interest is a change from a state in which mucus adheres to the region of interest to a state in which a non-neoplastic polyp appears in the region of interest.

20. An endoscope comprising:
the image processing apparatus according to claim 1; and
an endoscope main body to be inserted into a body including the observation target region.
